(19) United States
(12) Patent Application Publication
(10) Pub. No.: US 2012/0116041 A1
(43) Pub. Date: May 10, 2012

(54) REACTIVE SYSTEMS CONTAINING FORMAMIDES

(75) Inventors: Evelyn Peiffer, Leverkusen (DE); Harald Blum, Hafenlohr (DE)

(73) Assignee: BAYER MATERIALSCIENCE AG, LEVERKUSEN (DE)

(21) Appl. No.: 13/384,248

(22) PCT Filed: Jul. 6, 2010
(86) PCT No.: PCT/EP10/04107

§ 371 Date: Jan. 24, 2012

(30) Foreign Application Priority Data
Jul. 17, 2009 (DE) 10 2009 033 636.2

(51) Int. Cl. C08G 18/32; C07C 233/02

(52) U.S. Cl. 528/68; 564/192

(57) ABSTRACT

The invention relates to novel formamides derived from diamines or triamines. As compared to amines, these formamide-terminated compositions have a decelerated reactivity to polyisocyanates, that is, prepolymers.

Reactive amines are widely used in the paints, adhesives and sealants industry, inter alia, primarily as a crosslinker in reactive products for application as 2-component systems, e.g. in combination with polyisocyanates. The resulting crosslinked polyurethane polyureas are distinguished by a very good overall level of properties. Owing to the high reactivity of the amines, however, very rapid, sometimes spontaneous, reactions often occur, making safe, reproducible application difficult or even impossible.

Because of the high-quality properties that can be achieved with binder combinations of this type, there is still therefore a great need for amine components with retarded reactivity towards polyisocyanates or isocyanate-functional prepolymers, since this is essential to guarantee appropriate processing times (pot lives).

Reactive systems of this type containing formamides are hitherto unknown.

Surprisingly, it has been found that formamide-terminated compounds based on diamines or polyamines have retarded reactivity towards polyisocyanates or isocyanate-functional prepolymers compared with amines and can be processed e.g. into coatings, paints, adhesives, sealants, mouldings and foamed articles.

The present invention therefore provides novel reactive systems with—in comparison to amines—a prolonged processing time, which contain formamides.

The invention also provides reactive binder combinations containing

A) at least one component containing formamide structures
B) at least one component with polyisocyanate groups
C) optionally other components, optionally containing isocyanate-reactive groups.

Components A) containing formamide structures can be, for example,

A1) formamide-terminated, polyether-based oligomeric di- or polyamines, which react with polyisocyanates to form low-viscosity acyl urea prepolymers;
A2) other formamide-terminated oligo- and polyamines, e.g. amino-functional copolymers and amino-functional polycondensates;
A3) formamide-terminated low-molecular-weight compounds are those which can be obtained e.g. by the reaction of formic acid C1-C4 alkyl esters with amines.

Suitable amines are, for example mono-, di- and/or triamines with linear and/or branched and/or substituted and/or hetero atom-containing, e.g. oxygen atom-containing, aliphatic, cycloaliphatic, heterocyclic and/or aromatic structural units with 2 to 40, preferably 2 to 20 C atoms. They have a molecular weight of 45 to 700, preferably 60 to 300 g/mol.

As di- or triamines it is preferable to use aliphatic amines, e.g. ethylenediamine, 1,2-propylenediamine, 1,3-propylenediamine, 1,4-butanediamine, neopentanediamine, 1,5-di-amino-2-methylpentane (Dytek® A, DuPont), 2-butyl-2-ethyl-1,5-pentanediamine, 1,6-hexamethylenediamine, 2,5-diamino-2,5-dimethylhexane, 2,2,4- and/or 2,4,4-trimethyl-1,6-diaminohexane, 1,8-diaminooctane, 1,11-diaminoundecane, 1,12-diaminododecane, 4-aminomethyl-1,8-octanediamine (triaminononane), diethylenetriamine, triethylene-tetramine, cycloaliphatic amines such as e.g. 1-amino-3,3,5-trimethyl-5-aminomethyl-cyclohexane (isophorone diamine, IPDA), TCD diamine, 1,4-cyclohexanediamine, 2,4- and/or 2,6-hexahydrotoluenediamine (H₆TDA), isopropyl-2,4-diaminocyclohexane and/or isopropyl-2,6-diaminocyclohexane, tricyclodecanebis(methylamine), 1,3-bis(amino-methyl)cyclohexane, 2,4′- and/or 4,4′-diaminodicyclohexylmethane (PACM 20), 3,3′-dimethyl-4,4′-diaminodicyclohexylmethane (Laromin® C 260, BASF AG, DE), the isomeric diaminodicyclohexylmethanes having a methyl group as a ring substituent C-monomethyldiaminodicyclohexylmethane), 3(4)-aminomethyl-1-methylcyclohexylamine (AMCA) and araliphatic di- or triamines, such as e.g. 1,3-diaminobenzene, 1,4-diaminobenzene, 2,4- and/or 2,6-diaminotoluene (TDA), 1,3-bis(aminomethyl)benzene, 3,5-diethyltoluene-2,4-diamine, m-xylylenediamine, 4,6-dimethyl-1,3-benzene-dimethanamine, 4,4′- and/or 2,4′- and/or 2,2′-methylenebisbenzeneamine (MDA), or hetero-atom-containing amines dimer fatty acid diamine, bis(3-aminopropyl)methylamine, 4,9-dioxadodecane-1,12-diamine, 4,7,10-trioxamidecane-1,13-diamine, alkoxysilane-group-containing diamines. Also suitable are Michael adducts, which are obtained e.g. by the reaction of bifunctional primary amines with compounds containing unsaturated groups, such as e.g. hexanediol diacrylate etc.

Suitable polyisocyanate components B) can be polyisocyanates having at least two free isocyanate groups per molecule. Suitable examples are di- and polyisocyanates

X—(NCO)ₙ,

wherein n=2 to 10, preferably 2 to 5, and X denotes an aliphatic hydrocarbon residue with 4 to 36 carbon atoms, a cycloaliphatic hydrocarbon residue with 6 to 15 carbon atoms, an aromatic hydrocarbon residue with 6 to 15 carbon atoms or an araliphatic hydrocarbon residue with 7 to 15 carbon atoms.

Examples of these di- or polyfunctional polyisocyanates are 1,4-, 1,3-, and/or 1,2-cyclohexane diisocyanate, 1-methyl-2,4-diisocyanatocyclohexane, 1-methyl-2,6-diiso-cyanatocyclohexane, tetramethylene diisocyanate, octamethylene diisocyanate, decamethylene diisocyanate, dodecamethylene diisocyanate, H₆ 2,4- and/or 2,6-diisocyanatotoluene, 4,4′-diisocyanatodiphenylmethane, 2,4′-diisocyanatodiphenylmethane, 2,2′-diisocyanatodiphenylmethane, meta- and/or para-xylylene diisocyanate, 2,4-diisocyanatotoluene and/or 2,6-diisocyanatotoluene, isopropenyl dimethyltoluene diisocyanate, α,α,α,′α,′-tetramethyl m- and/or p-xylylene diisocyanate, 1,6-hexamethylene diisocyanate, trimethylhexane diisocyanate, tetramethylhexane diisocyanate, nonane triisocyanate, 1-isocyanato-3,3,5-trimethyl-5-isocyanatomethylcyclohexane (isophorone diisocyanate), 4,4′-diisocyanatodicyclohexylmethane and/or 2,4′-diisocyanato-dicyclohexylmethane and/or 2,2′-diisocyanatodicyclohexylmethane and the monomethyl- and dimethyl-substituted derivatives thereof.

Also suitable are reaction products, homologues, oligomers and/or polymers of the above-mentioned polyisocyanates with urethane, biuret, carbodiimide, isocyanurate, allophanate, iminooxadiazinedione and/or uretdione structural units, as well as mixtures of those mentioned as examples, optionally also with other isocyanates.

The average functionality of the polyisocyanate component B) is at least 1.5, preferably at least 2.0, particularly preferably at least 2.4.

The polyisocyanate component B) preferably consists of liquid oligomeric polyisocyanates based on hexamethylene diisocyanate, isophorone diisocyanate, H₆ 2,4- and/or 2,6-diisocyanatotoluene, 4,4′-diisocyanatodiphenylmethane, 2,4′-diisocyanatodiphenylmethane, 2,2′-diisocyanatodiphenylmethane, meta- and/or para-xylylene diisocyanate, 2,4-diisocyanatotoluene and/or 2,6-diisocyanatotoluene with urethane, urea, isocyanurate, biuret, uretdione, carbodiimide, allophanate and/or iminooxadiazinedione structural units and/or urethane and/or allophanate group-containing reaction products or prepolymers of the diisocyanates mentioned as preferred with hydroxy-functional compounds such as e.g. trimethylolpropane, butanediol, ethylene glycol, diethylene glycol, propylene glycol, neopentyl glycol, C2, C3 and/or C4 polyethers, polyesters, polycarbonates, castor oil.

The polyisocyanate component B) particularly preferably consists of hexamethylene diisocyanate, isophorone diisocyanate, 2,4-diisocyanatotoluene and/or 2,6-diisocyanatotoluene, 4,4′-diisocyanatodiphenylmethane, 2,4′-diisocyanatodiphenylmethane, 2,2′-diisocyanatodiphenylmethane or mixtures of isomers.

Components C) can be: hydroxy-, amino- and/or thiol-functional compounds, such as e.g. polyesters, C2 polyethers, C3 polyethers, C4 polyethers, polycarbonates, polyether carbonates, polymers, polycondensates, castor oil, polycaprolactones, alkyd resins, polyamines, polyamides, polyimides, polyvinyl acetates, polyvinyl alcohols, polyacrylates, polymethacrylates, polyolefins, copolymers, Michael adducts, polyepoxides and/or low-molecular-weight alcohols, amines and/or thiols, such as e.g. ethylene glycol, diethylene glycol, 1,2- or 1,3-propylene glycol, 1,4- or 1,3-butylene glycol, 1,6-hexanediol, 1,8-octanediol, neopentyl glycol, 1,4-hydroxymethylcyclohexane, 2-methyl-1,3-propanediol, glycerol, trimethylolpropane, 1,2,6-hexanetriol, 1,2,4-butanetriol, trimethylolethane, pentaerythritol, mannitol, sorbitol, methyl glycosides, sugars, phenol, isononylphenol, resorcinol, hydroquinone, 1,2,2- or 1,1,2-tris(hydroxyphenyl)ethane, ethylenediamine, tetra- or hexamethyleneamine, triethanolamine, aniline, phenylenediamine, 2,4- and 2,6-diaminotoluene and polyphenylpolymethylenepolyamine, isophorone diamine, diethyltoluenediamine (DETDA), 3,3′-dichloro-4,4′-diaminodiphenylmethane (MBOCA), 3,5-diamino-4-chloroisobutylbenzoate, 4-methyl-2,6-bis(methylthio)-1,3-diaminobenzene (Ethacure 300), trimethylene glycol di-p-aminobenzoate (Polacure 740M) and 4,4′-diamino-2,2′-dichloro-5,5′-diethyldiphenylmethane (MCDEA), the incorporation of free amines being limited to minor quantities.

Oxazolane-functional compounds, oxazolidine-functional compounds, aspartic acid esters, ketimines, aldimines, hexahydropyrimidines and/or tetrahydroimidazoles are also suitable as components C). It is also possible to use mixtures of the above-mentioned compounds C) and also compounds C) with different functional groups.

The invention also provides binder combinations based on formamides according to the invention which are present in crystalline or solid form at room temperature, and their use e.g. in or as powder coatings or hot-melt adhesives.

The production of the formamides can take place by a wide variety of methods:

The reaction of the di- and/or polyamines can take place in an excess of formic acid alkyl ester at the boiling point of the formic acid ester, with the excess formic acid alkyl ester and the alcohol that also results being distilled off at the end of the reaction of the amino group to form the formamide group.

It is also possible to react the mono-, di- or triamines to form the formamide-terminated low-molecular-weight compounds with formic acid or other formic acid derivatives, such as carbon monoxide, mixed formic acid-carboxylic acid anhydrides, low-molecular-weight amides or active esters of formic acid or preliminary reaction products of formic acid with amide coupling reagents, such as carbodiimides or condensed phosphoric acid derivatives.

It is also possible to react formamide, or the anion of formamide generated with a strong base, with alkylating reagents of formula (I)

X-[A]ₙ (I)

wherein X denotes an aliphatic, cycloaliphatic or aromatic residue, n denotes a natural number from 2 to 5 and A denotes a leaving group such as chloride, bromide, iodide, mesylate, tosylate or triflate.

The reaction to form formamide preferably takes place in an excess of formic acid C1-C4 alkyl ester, wherein one mole diamine is reacted with an excess of 2 to 6 moles formic acid C1-C4 alkyl ester, particularly preferably 2.5 to 4 moles, preferably methyl formate or ethyl formate, at the boiling point of the formic acid ester, the excess formic acid alkyl ester and the alcohol that also forms, preferably methanol or ethanol, being distilled off on completion of the reaction of the amino group to form the formamide group.

By mixing different amine components or solutions thereof, it is possible in this way to obtain a mixture of formamide components.

The reactive systems according to the invention can be cured from ambient temperature up to 250° C.

Catalysts that can be added to influence the reactivity are organometallic compounds such as tin(II) salts or titanium(IV) salts of carboxylic acids, strong bases such as alkali hydroxides, alcoholates and phenolates, e.g. di-n-octyltin mercaptide, dibutyltin maleate, diacetate, dilaurate, dichloride, bisdodecyl mercaptide, tin-II acetate, ethylhexanoate and diethylhexanoate, tetraisopropyl titanate or lead phenylethyl dithiocarbaminate. Another class of compounds is represented by the dialkyltin(IV) carboxylates. It is also possible to use dicarboxylic acids. The following may be mentioned as examples of acids: adipic acid, maleic acid, fumaric acid, malonic acid, succinic acid, pimelic acid, terephthalic acid, phenylacetic acid, benzoic acid, acetic acid, propionic acid and 2-ethylhexanoic, caprylic, capric, lauric, myristic, palmitic and stearic acids. Specific compounds are dibutyl- and dioctyltin diacetate, maleate, bis(2-ethylhexanoate), dilaurate, tributyltin acetate, bis(β-methoxycarbonylethyl)tin dilaurate and bis(β-acetyl ethyl)tin dilaurate.

Tin oxides and sulfides as well as thiolates can also be used. Specific compounds are: bis(tributyltin) oxide, bis(trioctyltin) oxide, dibutyl- and dioctyltin bis(2-ethylhexyl thiolate) dibutyl- and dioctyltin didodecyl thiolate, bis(β-methoxycarbonylethyl)tin didodecyl thiolate, bis(β-acetyl ethyl)tin bis(2-ethylhexyl thiolate), dibutyl- and dioctyltin didodecyl thiolate, butyl- and octyltin tris(thioglycolic acid 2-ethylhexanoate), dibutyl- and dioctyltin bis(thioglycolic acid 2-ethylhexanoate), tributyl- and trioctyltin (thioglycolic acid 2-ethylhexanoate) as well as butyl- and octyltin tris(thioethylene glycol 2-ethylhexanoate), dibutyl- and dioctyltin bis(thioethylene glycol 2-ethylhexanoate), tributyl- and trioctyltin (thioethylene glycol 2-ethylhexanoate), bis(β-methoxycarbonylethyl)tin bis(thioethylene glycol 2-ethylhexanoate), bis(β-methoxycarbonylethyl)tin bis(thioglycolic acid 2-ethyl-hexanoate) and bis(β-acetyl ethyl)tin bis(thioethylene glycol 2-ethylhexanoate) and bis(β-acetyl ethyl)tin bis(thioglycolic acid 2-ethylhexanoate).

Organobismuth compounds, e.g. triarylbismuth compounds, oxides of these compounds and alkyl or arylhalobismuthines of the types R2 BiX and R3 BiX2, as well as phenolates and carboxylates of bismuth, can also be used. In particular, bismuth carboxylates are used as organobismuth compounds, the carboxylic acids possessing 2 to 20 C atoms, preferably 4 to 14 C atoms. The following acids are mentioned specifically: butyric acid, caproic acid, caprylic acid, capric acid, lauric acid, myristic acid, palmitic acid, stearic acid, arachic acid, isobutyric acid and 2-ethylhexanoic acid. Mixtures of bismuth carboxylates with other metal carboxylates, e.g. tin carboxylates, can also be used.

In particular the following tertiary amines are used as catalyst, individually or in combination with at least one of the above-mentioned catalysts: diazabicyclooctane (Dabco), triethylamine, dimethylbenzylamine (Desmorapid DB, Bayer), bisdimethylaminoethyl ether (Catalyst Al, UCC), tetramethylguanidine, bisdimethylaminomethyl phenol, 2,2′-dimorpholinodiethyl ether, 2-(2-dimethylaminoethoxy)ethanol, 2-di-methylaminoethyl-3-dimethylaminopropyl ether, bis(2-dimethylaminoethyl)ether, N,N-dimethylpiperazine, N-(2-hydroxyethyl)-2-azanorborane, Tacat® DP-914 (Texaco Chemical), Jeffcat®, N,N,N,N-tetramethylbutane-1,3-diamine, N,N,N,N-tetramethyl-propane-1,3-diamine, N,N,N,N-tetramethylhexane-1,6-diamine as well as, for example, triethanolamine or triisopropanolamine.

The catalysts may also be present in oligomerised or polymerised form, e.g. as N-methylated polyethyleneimine.

Also suitable are 1-methylimidazole, 2-methyl-1-vinylimidazole, 1-allylimidazole, 1-phenylimidazole, 1,2,4,5-tetramethylimidazole, 1-(3-aminopropyl)imidazole, pyrimidazole, 4-dimethylaminopyridine, 4-pyrrolidinopyridine, 4-morpholinopyridine, 4-methylpyridine and N-dodecyl-2-methylimidazole.

To achieve special effects, it is also possible to add small amounts of auxiliary substances that are conventional in the paints and adhesives industry during production of the reactive systems according to the invention, such as e.g. surface-active substances, emulsifiers, stabilisers, anti-settling agents, UV stabilisers, catalysts for the crosslinking reaction, defoamers, antioxidants, anti-skinning agents, flow promoters, thickeners and/or bactericides.

The reactive systems according to the invention can be used in or as a paint, coating, size, ink, printing ink, adhesive, sealant, hot-melt adhesive, bonding foam, laminating adhesive, encapsulating compound, flexible, rigid or structural foam for coating, bonding, sealing of optionally already pre-coated mineral or ceramic substrates and materials, concrete, asphalt, bitumen, hard fibre materials, metallic substrates, plastics, paper, printing paper, card, composite materials, glass, china, textiles, leather, wooden and wood-like substrates such as e.g. furniture, fibreboards, parquet, window frames, doors, fences, panels, boards, beams, roofs, e.g. as a one-coat paint, in multi-coat paints, as a priming coat, intermediate coat, filler, basecoat, topcoat, barrier layer, primer, adhesion promoter, protective coat, strip lacquer, temporary coating, size, in functional coats, as overcoats, clear lacquer, pigmented lacquer, for the production of mouldings and, in addition, also in adhesives, sealants, printing inks, inks, foams, films and fibres.

The production of the coating can take place by the various spray methods, such as e.g. compressed air, airless or electrostatic spray methods, using one- or optionally two-component spray equipment. The paints and coating compositions to be produced and used according to the invention can also be applied by other methods, however, e.g. by brushing, rolling or knife coating.

### EXAMPLES

Starting materials used:

1,6-Hexamethylenediamine, a bifunctional, amino-terminated, aliphatic compound,
2-Methyl-1,5-diaminopentane, a bifunctional, amino-terminated, branched, aliphatic compound,
Jeffamin® ED 600 (Huntsman, UK) a bifunctional polyether amine, 4,7,10-Trioxamidecane-1,13-diamine, a bifunctional, amino-terminated compound,
4-Aminomethyl-1,8-octanediamine, a trifunctional, amino-terminated, aliphatic compound,
Ethyl acetate,
Benzoyl chloride,
Irganox® 1076 (Ciba, CH), a sterically hindered phenol,
Desmodur® N 3300 (Bayer MaterialScience AG, Germany), an aliphatic polyisocyanate with an NCO content of 21.8%,
Desmodur® N 100, an aliphatic polyisocyanate with an NCO content of 22.0%,
Desmodur® E 23, an aromatic prepolymer with an NCO content of 15.4%,
Desmodur® E 14, an aromatic prepolymer with an NCO content of 3.3%,
Desmodur® E 14, an aliphatic-aromatic prepolymer with an NCO content of 10.5%.
Pot life: time between production of the mixture and clear increase in viscosity or crosslinking (pot life).
Solvent resistance: a solvent-impregnated pad is applied for 1 minute on to the surface to be tested. After removal, the surface is visually inspected and evaluated.

### Example 1

At a maximum of 50° C., 222 g ethyl formate are added dropwise within 4 h to 116 g 1,6-hexamethylenediamine dissolved in 170 g ethanol and stirring is continued for 4 h. The excess ethyl formate and the ethanol that has been formed and used are then distilled off.

A formamide-terminated low-molecular-weight compound is obtained with a melting point of 105-108° C. and reactivities as set out in Table 1.

### Example 2

At a maximum of 50° C., 222 g ethyl formate are added dropwise within 4 h to 116 g 2-methyl-1,5-diaminopentane and stirring is continued for 4 hours. The excess ethyl formate and the ethanol that has been formed are then distilled off.

A formamide-terminated low-molecular-weight compound is obtained with a viscosity of 581 mPas and reactivities as set out in Table 1.

### Example 3

At a maximum of 50° C., 222 g ethyl formate are added dropwise within 2 hours to 600 g Jeffamin® ED 600 and stirring is continued for 4 hours under reflux. The excess ethyl formate and the ethanol that has been formed are then distilled off.

A formamide-terminated oligomeric compound is obtained with a viscosity of 223 mPas and reactivities as set out in Table 1.

### Example 4

At a maximum of 50° C., 800 g ethyl formate are added dropwise within 3 hours to 793 g 4,7,10-trioxamidecane-1,13-diamine and stirring is continued for 4 hours under reflux. The excess ethyl formate and the ethanol that has been formed and used are then distilled off.

A formamide-terminated low-molecular-weight compound is obtained with a viscosity of 239 mPas and reactivities as set out in Table 1.

### Example 5

At a maximum of 50° C., 667 g ethyl formate are added dropwise within 3 hours to 346 g 4-aminomethyl-1,8-octanediamine and stirring is continued for 4 hours under reflux. The excess ethyl formate and the ethanol that has been formed and used are then distilled off.

A formamide-terminated low-molecular-weight compound is obtained with a viscosity of 10200 mPas and reactivities as set out in Table 1.

Comparison Substances

### 1. Hexanediamine

2. 2-Methyl-1,5-diaminopentane
3. Aspartic ester based on 2 mol dimethyl maleate and 1 mol 2-methyl-1,5-diaminopentane

In Table 1 (following page) pot lives and some paint properties of the reactive systems according to the invention based on formamides are compared with those based on conventional amines. The reactive systems according to the invention containing formamides all have significantly longer processing times/pot lives than the comparable amines. Practical processing times are achieved.

| Polyisocyanate | | | | | | | | |
|---|---|---|---|---|---|---|---|---|
| crosslinker | Ex. 1 | Ex. 2 | Ex. 3 | Ex. 4 | Ex. 5 | Comp. 1 | Comp. 2 | Comp. 3 |
| Desmodur ® | | Pot life > 5 h | Pot life > 5 h | Pot life 80 min | Pot life > 7 h | Pot life < 2 s | Pot life < 2 s | Pot life 15 min |
| N 3300 | | clear film | | | | | | |
| | | smooth surface | | | | | | |
| | | good ethanol | | | | | | |
| | | resistance | | | | | | |
| | | good xylene | | | | | | |
| | | resistance | | | | | | |
| Desmodur ® | Pot life 2 min | Pot life > 5 h | Pot life > 5 h | Pot life 90 min | Pot life > 7 h | Pot life < 2 s | Pot life < 2 s | Pot life 15 min |
| N100 | clear film | | | | | | | |
| | smooth | | | | | | | |
| | surface | | | | | | | |
| Desmodur ® | Pot life 2 min | Pot life 2 h | Pot life 2 h | Pot life 3 h | Pot life 3 h | Pot life < 2 s | Pot life < 2 s | |
| E 23 | | | clear film | | | | | |
| | | | rubber-like | | | | | |
| | | | surface | | | | | |
| | | | pendulum | | | | | |
| | | | hardness 17 s | | | | | |
| Desmodur ® | | Pot life 50 min | Pot life 75 min | Pot life 75 min | Pot life 7 h | Pot life < 2 s | Pot life < 2 s | |
| HL | | clear film | clear film | clear film | clear film | | | |
| | | smooth surface | smooth surface | smooth surface | smooth surface | | | |
| | | pendulum | pendulum | pendulum | pendulum | | | |
| | | hardness 90 s | hardness 30 s | hardness 97 s | hardness 66 s | | | |
| | | good ethanol | good ethanol | good ethanol | good ethanol | | | |
| | | resistance | resistance | resistance | resistance | | | |
| | | good xylene | good xylene | good xylene | good xylene | | | |
| | | resistance | resistance | resistance | resistance | | | |
| Desmodur ® | Pot life 2 min | Pot life 5 h | Pot life > 5 h | Pot life > 5 h | Pot life > 7 h | Pot life < 2 s | Pot life < 2 s | |
| E14 | | clear film | | | | | | |
| | | rubber-like | | | | | | |
| | | surface | | | | | | |
| | | pendulum | | | | | | |
| | | hardness 49 s | | | | | | |

1.-10. (canceled)

11. A reactive binder system comprising at least one formamide.

12. The reactive binder system according to claim 11, wherein the reactor binder system comprises
A) at least one component comprising formamide structures,
B) at least one component comprising polyisocyanate groups and
C) optionally other components, optionally comprising isocyanate-reactive groups.

13. The reactive binder systems according to claim 12, wherein component A) comprises at least one formamide based on di- and/or triamines, which contain 2 to 40 carbon atoms.

14. The reactive binder system according to claim 12, wherein component A) comprises at least one formamide based on polyether amines, which contain 2 to 40 carbon atoms.

15. A paint or coating composition comprising the reactive binder system according to claim 11.

16. An adhesive or sealant composition comprising the reactive binder system according to claim 11.

17. An ink comprising the reactive binder system according to claim 11.

18. The ink according to claim 17, wherein the ink is a printing ink.

19. A flat or foamed moulding comprising the reactive binder system according to claim 11.

20. A size comprising the reactive binder system according to claim 11.

21. A method for coating, bonding and/or sealing a substrate comprising contacting a composition comprising the reactive binder system according to claim 11 with the substrate, wherein the substrate is selected from the group consisting of metal, wood, timber-based materials, leather, textiles, plastics, mineral materials, cork, fibres, concrete, paper, card and films.
